(19) United States
(12) Patent Application Publication
(10) Pub. No.: US 2019/0224169 A1
(43) Pub. Date: Jul. 25, 2019

(54) Methods of Treatment of Hypertriglyceridemia

(71) Applicant: Kowa Company, Ltd., Nagoya-shi (JP)

(72) Inventors: Ryu Oshima, Tokyo (JP); Kazuhito Suehira, Tokyo (JP); Gary Gordon, Morrisville, NC (US)

(21) Appl. No.: 16/227,440

(22) Filed: Dec. 20, 2018

Related U.S. Application Data
(60) Provisional application No. 62/609,048, filed on Dec. 21, 2017.

(51) Int. Cl. A61K 31/4168; A61P 3/06

(52) U.S. Cl. CPC A61K 31/4168; A61P 3/06

(57) ABSTRACT

The present invention relates to pharmacological interventions with pemafibrate for moderate or severe hypertriglyceridemia.

### FIELD OF THE INVENTION

The present invention relates to pharmacological interventions with pemafibrate for moderate hypertriglyceridemia (serum TG≥200 mg/dL and <500 mg/dL) or severe hypertriglyceridemia (serum TG≥500 mg/dL).

### BACKGROUND OF THE INVENTION

A variety of primary disorders of lipoprotein metabolism have been described which may lead to elevated levels of the atherogenic lipoproteins (very low-density lipoprotein (VLDL), remnant particles, low-density lipoprotein (LDL), etc.) or reduced levels of the anti-atherogenic high-density lipoprotein, any or all of which can confer increased risk of coronary artery disease. Of greater concern, elevated levels of triglyceride (TG), in particular TG levels≥500 mg/dL (5.65 mmol/L), confer an increased risk of acute pancreatitis.^{1,2} Acute pancreatitis caused by hypertriglyceridemia (HTG) is associated with increased severity and rates of complications compared to pancreatitis with causes other than HTG.^{3,4}

Fibrates improve TG and high-density lipoprotein cholesterol (HDL-C) by activating peroxisome proliferator-activated receptor alpha (PPARα),⁵ and are approved in the United States for the treatment of severe HTG. In the United States, fenofibrate, fenofibric acid, and gemfibrozil are available. Fibrates available in Europe are bezafibrate, ciprofibrate, fenofibrate, and gemfibrozil. In Japan, bezafibrate, clinofibrate, clofibrate, and fenofibrate are available.

The United States Adult Treatment Panel III National Cholesterol Education Program (NCEP) guidelines⁶ recommend reduction of TG through lifestyle, diet, and pharmacologic methods as the first priority of therapy when serum TG are ≥500 mg/dL. Treatment with omega-3 fatty acids, such as those found in fish oils, has been shown to effectively decrease TG levels up to 30%; however, for individuals with severe HTG, increasing omega-3 fatty acid intake does not adequately manage TG levels.⁷

The European Society for Cardiology and European Atherosclerosis Society consensus guidelines note that patients can develop pancreatitis with TG concentrations between 5 and 10 mmol/L (440 and 880 mg/dL).⁸ These guidelines also recommend initiating fibrates to prevent acute pancreatitis.

Most fibrates are contraindicated or require careful administration in patients with renal dysfunction. Furthermore, coadministration of these drugs with statins is contraindicated in patients with severe renal dysfunction. Thus, there are restrictions on the use of existing PPARα agonists.^{9,10,11,12,13,14,15,16}

Pemafibrate, whose chemical name is (2R)-2-[3-({1,3-benzoxazol-2-yl[3-(4-methoxyphenoxy)propyl]amino}methyl)phenoxy]butanoic acid, is a PPARα activator like fenofibrate, although it has proven much more potent at affecting lipid metabolism and is more specific for the PPARα receptor than fenofibrate. Thus, pemafibrate is also described as a selective PPARα modulator (SPPARMα). The drug was recently approved for the treatment of hyperlipidemia in Japan and is under development for the treatment of cardiovascular disease world-wide.

Pemafibrate is approximately 2500 times more active than fenofibric acid in terms of the EC₅₀ of the PPARα-activating effect. It is more potent than fenofibrate in decreasing TG and increasing HDL-C in apolipoprotein (Apo) A1 transgenic mice. In previous clinical trials, pemafibrate has been administered at doses ranging from 0.1 mg to 1.6 mg per day in healthy adults. Doses up to 0.4 mg per day have been administered in patients with dyslipidemia. Pemafibrate demonstrated dose-dependent decreases in TG in both Japanese and European patients. In study K-877-201, a Phase 2 dose-finding study conducted in Europe, pemafibrate 0.2 mg taken twice daily demonstrated the greatest efficacy with a placebo-adjusted TG reduction of 54.4%. Greater efficacy in TG reduction and HDL-C elevation was observed when pemafibrate was administered twice daily compared to once daily. Treatment with pemafibrate also resulted in changes in the following lipid parameters from baseline to Week 12 with last observation carried forward as determined in the analysis of secondary efficacy endpoints: increases in Apo A1, Apo A2, fibroblast growth factor 21 (FGF21), HDL-C; and decreases in Apo B48, Apo C2, Apo C3, and VLDL-C. Increases in LDL-C, both by beta quantification and calculation with the Friedewald equation, were also observed. Based on analysis of efficacy variables, pemafibrate appeared to have a lowering effect on Apo C3 that led to the conversion of VLDL particles to LDL particles, increasing the fraction of larger LDL particles, and reducing TG levels. Similarly, the reduction of Apo C3 led to increased removal of remnant-like particles and lowering of Apo B48. Overall, no changes were observed in total Apo B levels, indicating that the increase in LDL-C was not associated with an increase in LDL particle number or coronary heart disease (CHD) risk, which was supported by the observed decrease in non-HDL-C, a parameter that more accurately reflects CHD risk than LDL-C. The observed increases in Apo A1 and HDL-C resulted from both increased production of Apo A1 in the liver and increased turnover of TG-rich lipoproteins, both of which were associated with a decreased CHD risk.

Beyond this information, the effect of pemafibrate on the patient with moderate and severe hypertriglyceridemia is unknown. Furthermore, the effect of pemafibrate on the patient with renal impairment along with moderate or severe hypertriglyceridemia, particularly when combined with a statin, is also unknown.

It is therefore an object of the present invention to provide pemafibrate therapies that can treat patients with moderate or severe hypertriglyceridemia along with normal renal function or renal impairment.

### SUMMARY OF THE INVENTION

The invention relates to the surprising ability of pemafibrate to reduce plasma triglyceride even in patients with moderate and severe hypertriglyceridemia. Thus, the invention provides a method of treating moderate and severe hypertriglyceridemia in a subject in need thereof, comprising administering to the subject a therapeutically effective amount of pemafibrate or a pharmaceutically acceptable salt thereof. The invention also provides a method of treating moderate or severe hypertriglyceridemia in a subject in need thereof, wherein the patient also is renally impaired, comprising administering to the patient a therapeutically effective amount of pemafibrate or a pharmaceutically acceptable salt thereof.

### DETAILED DESCRIPTION OF THE INVENTION

The present invention provides the following:
1) A method of treating moderate or severe hypertriglyceridemia in a subject in need thereof, comprising administering to the patient a therapeutically effective amount of pemafibrate or a pharmaceutically acceptable salt thereof.
2) A method of treating severe hypertriglyceridemia in a subject in need thereof, comprising: (a) identifying a subject having a fasting baseline triglyceride level of about 500 mg/dl (5.65 mmol/L) and over, and (b) administering to the subject a pharmaceutical composition comprising pemafibrate or a pharmaceutically acceptable salt thereof.
3) A method of treating severe hypertriglyceridemia in a subject in need thereof, comprising: (a) identifying a subject having a fasting baseline triglyceride level of about 500 mg/dl (5.65 mmol/L) to about 2000 mg/dl (22.6 mmol/L), and (b) administering to the subject a pharmaceutical composition comprising pemafibrate or a pharmaceutically acceptable salt thereof.
4) The method according to any one of 1) to 3), wherein the therapeutically effective amount of pemafibrate or pharmaceutically acceptable salt thereof is from 0.2 to 1.0 mg, administered orally per day.
5) The method according to any one of 1) to 3), wherein the therapeutically effective amount of pemafibrate or pharmaceutically acceptable salt thereof is 0.4 mg, administered orally per day.
6) The method according to 5), wherein the therapeutically effective amount of pemafibrate or pharmaceutically acceptable salt thereof is administered twice daily.
7) The method according to any one of 1) to 3), wherein the therapeutically effective amount of pemafibrate or a pharmaceutically acceptable salt thereof is 0.2 mg, administered orally per day.
8) The method according to 7), wherein the therapeutically effective amount of pemafibrate or pharmaceutically acceptable salt thereof is administered twice daily.
9) The method according to any one of 1) to 3), wherein the patient has normal renal function.
10) The method according to any one of 1) to 3), wherein the patient has mild or moderate renal impairment.
11) The method according to any one of 1) to 8), wherein the patient has severe renal impairment.
12) The method of any one of 1) to 11), wherein the patient is on high intensity statin therapy and aged≥21 years with clinical ASCVD selected from a history of acute coronary syndrome or myocardial infarction, stable or unstable angina, coronary revascularization, stroke, transient ischemic attack [TIA] of atherosclerotic origin, or peripheral arterial disease or revascularization.
13) The method of any one of 1) to 11), wherein the patient is on high intensity statin therapy and aged≥21 years with a history of LDL-C≥190 mg/dL, which is not due to secondary modifiable causes.
14) The method of any one of 1) to 11), wherein the patient is on moderate or high intensity statin therapy and aged 40 to 75 years, inclusive, without clinical ASCVD but with type-2 diabetes and a history of LDL-C of 70 to 189 mg/dL, inclusive.
15) The method of any one of 1) to 11), wherein the patient is on moderate or high intensity statin therapy and aged 40 to 75 years, inclusive, without clinical ASCVD or diabetes, with a history of LDL-C of 70 to 189 mg/dL, inclusive, with estimated 10-year risk for ASCVD of ≥7.5% by the Pooled Cohort Equation.
16) The method of any one of 1) to 15), wherein the subject has one or a combination of low HDL-C levels, elevated LDL-C levels, elevated non-HDL-C levels, or elevated Total Cholesterol levels.
17) The method of any one of 1) to 16), wherein said subject is an adult and not on statin therapy, comprising administering 0.2 mg of pemafibrate or a pharmaceutically acceptable salt thereof twice daily to said subject, further comprising administering 0.2 mg of pemafibrate or a pharmaceutically acceptable salt thereof to a second adult subject on moderate or high intensity statin therapy with moderate or severe hypertriglyceridemia.
18) The method of 17), wherein said second adult subject is on high intensity statin therapy.
19) The method of any one of 1) to 17) wherein said subject is an adult and not renally impaired, comprising administering 0.2 mg of pemafibrate or a pharmaceutically acceptable salt thereof twice daily to said subject, further comprising administering 0.2 mg of pemafibrate or a pharmaceutically acceptable salt thereof to a second adult subject who is renally impaired with moderate or severe hypertriglyceridemia.
20) The method of 19), wherein said second subject has mild to moderate renal impairment.
21) The method of any one of 1) to 20), wherein the subject has a fasting baseline triglyceride level of greater than 750 mg/dl (8.475 mmol/L).
22) The method of any one of 1) to 20), wherein the subject has a fasting baseline triglyceride level of greater than 1000 mg/dl (11.3 mmol/L).
23) The method of any one of 1) to 20), wherein the subject has a fasting baseline triglyceride level of greater than 1500 mg/dl (16.95 mmol/L).
24) A method of treating dyslipidemia in a renally impaired adult patient and a non-renally impaired adult patient comprising administering to both patients 0.2 mg of pemafibrate or a pharmaceutically acceptable salt thereof twice daily.
25) The method of 24) wherein the renally impaired patient is mildly to moderately renally impaired.
26) The method of 24) wherein the renally impaired patient is severely renally impaired.
27) The method of any one of 1) to 26) wherein the subject or renally impaired patient has an HDL-C concentration of less than 40 mg/dL.
28) The method of any one of 1) to 26) wherein the subject or renally impaired patient is on moderate or high intensity statin therapy.
29) The method of any one of 1) to 26) wherein the subject has an LDL-C concentration less than 70 mg/dL.
30) The method of any one of 1) to 26) wherein the subject or renally impaired patient has an HDL-C concentration of less than 40 mg/dL and is on moderate to high intensity statin therapy.
31) The method of any one of 1) to 26) wherein the subject or renally impaired patient has an HDL-C concentration of less than 40 mg/dL and has an LDL-C concentration less than 70 mg/dL.
32) The method of 1) wherein the subject or renally impaired patient has an HDL-C concentration of less than 40 mg/dL and is on moderate to high intensity statin therapy and has a triglyceride concentration of from 200 to 500 mg/dL.
33) The method of 1) wherein the subject or renally impaired patient has an HDL-C concentration of less than 40 mg/dL and has an LDL-C concentration less than 70 mg/dL and has a triglyceride concentration of from 200 to 500 mg/dL.
34) The method of any one of 1) to 33) wherein the method prevents a cardiovascular event selected from nonfatal myocardial infarction, nonfatal ischemic stroke, hospitalization for unstable angina requiring unplanned coronary revascularization, cardiovascular death, and combinations thereof.

As used in the specification and claims, the singular forms “a,” “an,” and “the” include plural references unless the context clearly dictates otherwise. For example, the term “a pharmaceutical excipient” refers to one or more pharmaceutical excipients for use in the presently disclosed formulations and methods.

The use of numerical values in the various quantitative values specified in this application, unless expressly indicated otherwise, are stated as approximations as though the minimum and maximum values within the stated ranges were both preceded by the word “about”. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

As used herein, “therapeutically effective amount” refers to an amount sufficient to elicit the desired biological response in a patient. The therapeutically effective amount or dose depends on the age, sex and weight of the patient, and the current medical condition of the patient. The skilled artisan can determine appropriate amount or dose depending on the above factors based on his or her knowledge and the teachings contained herein.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. “Pharmaceutically acceptable salts” means salts that are pharmaceutically acceptable, as defined above, and which possess the desired pharmacological activity.

The terms “treating” and “treatment,” when used herein, refer to the medical management of a patient with the intent to cure, ameliorate, stabilize, or prevent a disease, pathological condition, or disorder (collectively “disorder”). These terms include active treatment, that is, treatment directed specifically toward the improvement of a disorder, and also include causal treatment, that is, treatment directed toward removal of the cause of the associated disorder. In addition, this term includes palliative treatment, that is, treatment designed for the relief of symptoms rather than the curing of the disorder; preventative treatment, that is, treatment directed to minimizing or partially or completely inhibiting or delaying the development of the disorder; and supportive treatment, that is, treatment employed to supplement another specific therapy directed toward the improvement of the disorder.

All analyte measurements recited herein, when used to define a patient described herein, are measured at the beginning of pemafibrate treatment.

Unless stated herein to the contrary, all analyte measurements are taken in the fasting state, and are based on the concentration of the analyte in plasma or serum. The fasting state means that the patient has not eaten anything in from 8 to 12 hours, except for water. Standard methods of measuring analytes can be found in Lab Protocols for NHANES 2003-2004 data published by the United States Centers for Disease Control.

Unless stated herein to the contrary, all methods described herein are performed in all ages, preferably greater than 18 years.

As used herein, the term “significantly” refers to a level of statistical significance. The level of statistical significant can be, for example, of at least p<0.05, of at least p<0.01, of at least p<0.005, or of at least p<0.001.

As used herein, the term “normal renal function” refers to a situation in which the renal function of the patient of this invention is normal. In general, an estimated glomerular filtration rate (eGFR) of 90 mL/min/1.73 m² or more (eGFR≥90) qualifies as normal renal function.

As used herein, the term “mild renal impairment” refers to a situation in which the renal function of the patient of this invention is mildly impaired. In general, an eGFR less than 90 mL/min/1.73 m² and greater than or equal to 60 mL/min/1.73 m² (60≤eGFR<90) qualifies as mild renal impairment.

As used herein, the term “moderate renal impairment” refers to a situation in which the renal function of the patient of this invention is moderately impaired. In general, an eGFR less than 60 mL/min/1.73 m² and greater than or equal to 30 mL/min/1.73 m² (30≤eGFR<60) qualifies as moderate renal impairment.

As used herein, the term “mild or moderate renal impairment” refers to a situation in which the renal function of the patient of this invention is mildly or moderately impaired. In general, an eGFR less than 90 mL/min/1.73 m² and greater than or equal to 30 mL/min/1.73 m² (30≤eGFR<90) qualifies as mild or moderate renal impairment.

As used herein, the term “severe renal impairment” refers to a situation in which the renal function of the patient of this invention is severely impaired. In general, an eGFR less than 30 mL/min/1.73 m² (eGFR<30) qualifies as severe renal impairment.

ASCVD when used herein refers to atherosclerotic cardiovascular disease.

The “Pooled Cohort Equation” is reported at Preiss D, Kristensen S L, The new pooled cohort equations risk calculator. CAN J CARDIOL. 2015 May; 31(5):613-9.

Statins, also known as HMG-CoA reductase inhibitors, include atorvastatin, simvastatin, fluvastatin, pitavastatin, rosuvastatin, pravastatin, and lovastatin and their pharmaceutically acceptable salts. Statins are generally classified as high, moderate or low intensity, based on the degree of LDL-C reduction they have demonstrated in controlled clinical trials, as summarized in the following table derived from ACC/AHA Release Updated Guideline on the Treatment of Blood Cholesterol to Reduce ASCVD Risk, AMERICAN FAMILY PHYSICIAN, Volume 90, Number 4 (Aug. 15, 2014):

| High intensity | Moderate intensity | Low intensity |
|---|---|---|
| Daily dosage lowers LDL-C | Daily dosage lowers LDL-C | Daily dosage lowers LDL-C |
| by approximately ≥50% | by approximately 30% to 50% | by approximately <30% |
| on average | on average | on average |
| Atorvastatin, 40 to 80 mg | Atorvastatin, 10 (20) mg | Simvastatin, 10 mg |
| Rosuvastatin, 20 (40) mg | Rosuvastatin, (5) 10 mg | Pravastatin, 10 to 20 mg |
| | Simvastatin, 20 to 40 mg | Lovastatin, 20 mg |
| | Pravastatin, 40 (80) mg | Fluvastatin, 20 to 40 mg |
| | Lovastatin, 40 mg | Pitavastatin, 1 mg |
| | Fluvastatin XL, 80 mg | |
| | Fluvastatin, 40 mg twice daily | |
| | Pitavastatin, 2 to 4 mg | |

NOTE:
Specific statins and dosages noted in bold were evaluated in RCTs included in critical question 1, critical question 2, and the Cholesterol Treatment Trialists 2010 meta-analysis included in critical question 3 (see full guideline for details). All of these RCTs demonstrated a reduction in major cardiovascular events. Statins and dosages listed in italics are approved by the U.S. Food and Drug Administration but were not tested in the RCTs reviewed.
RCT = randomized clinical trial.

When the term “moderate to high intensity statin therapy” is employed, the following group of statin therapies is preferably administered, and can be substituted for the term “moderate to high intensity statin therapy”: atorvastatin≥40 mg/day (based on the weight of the free base), rosuvastatin≥20 mg/day (based on the weight of the calcium salt), and simvastatin≥40 mg/day (based on the weight of the free base), or pitavastatin≥4 mg/day. The term “non-moderate to high intensity statin therapy” refers to any statin therapy other than atorvastatin≥40 mg/day (based on the weight of the free base), rosuvastatin≥20 mg/day (based on the weight of the calcium salt), and simvastatin≥40 mg/day (based on the weight of the free base), or pitavastatin≥4 mg/day.

As used herein, a patient tested for a biomarker that is “elevated” or “low” means that the patient is at risk for an adverse cardiovascular event. The Third Report of the National Cholesterol Education Program (“NCEP”) Expert Panel on Detection, Evaluation, and Treatment of High Blood Cholesterol in Adults (Adult Treatment Panel III) discloses lipid cut-points for evaluating cardiovascular risk. Under these cut-points, a person having an LDL-C concentration greater than 100 mg/dL (2.59 mmol/L) or even 70 mg/dL (1.81 mmol/L) is at risk for a cardiovascular event. A person having a total cholesterol concentration greater than 200 mg/dL (5.18 mmol/L) is at risk for a cardiovascular event. A person having an HDL-C concentration less than 40 mg/dL (1.0 mmol/L) for men and less than 50 mg/dL (1.3 mmol/L) for women is at risk for a cardiovascular event. A person having a fasting triglyceride concentration greater than 200 mg/dL (2.27 mmol/dL) or even 150 mg/dL (1.70 mmol/L) is at risk for cardiovascular events. A person having a non-HDL-C concentration greater than 130 mg/dL (3.37 mmol/L) is also at risk for a cardiovascular event.

While the methods of the present invention are particularly useful in the treatment of elevated triglycerides, they also are useful for the treatment of patients with one or a combination of low HDL-C levels, elevated LDL-C levels, elevated non-HDL-C levels, or elevated Total Cholesterol levels. Thus, the methods are also useful for the treatment of patients with:
low HDL-C
elevated LDL-C
elevated non-HDL-C
elevated Total Cholesterol
low HDL-C and elevated LDL-C
low HDL-C and elevated non-HDL-C
low HDL-C and elevated Total Cholesterol
low HDL-C and elevated LDL-C and elevated non-HDL-C
low HDL-C and elevated LDL-C and elevated Total Cholesterol
low HDL-C and elevated non-HDL-C and elevated Total Cholesterol
low HDL-C and elevated LDL-C and elevated non-HDL-C and elevated Total Cholesterol
elevated LDL-C and elevated non-HDL-C
elevated LDL-C and elevated Total Cholesterol
elevated LDL-C and elevated non-HDL-C and elevated Total Cholesterol
elevated non-HDL-C and elevated Total Cholesterol

Total pemafibrate daily doses ranging from 0.1 mg to 0.4 mg, administered daily or divided twice daily, have demonstrated an acceptable safety profile. Because the urinary excretion of pemafibrate is low, as shown by nonclinical and clinical studies, it is expected that pemafibrate can be used safely even in patients with renal impairment. In addition, because the drug has minimal inhibitory effects on major drug metabolizing enzymes in the liver, it is unlikely to cause drug-drug interactions; however, drugs which are strong organic anion-transporting polypeptide (OATP) inhibitors (e.g., cyclosporine and rifampin) do interact with pemafibrate. Therefore, pemafibrate is expected to exhibit not only a potent lipid metabolism-improving effect but also to serve as a drug with a broad therapeutic range with fewer restrictions in patients with renal dysfunction or with concomitant drugs than existing PPARα agonists.

Data from study K-877-12 in Japanese patients with renal impairment found no meaningful differences in pemafibrate pharmacokinetics (PK), even in patients with severe renal impairment, suggesting adjustment of dosing for renal impairment will not be necessary with pemafibrate to ensure patient safety.

In a first embodiment, the invention provides a method of treating moderate or severe hypertriglyceridemia in a subject in need thereof, comprising administering to the patient a therapeutically effective amount of pemafibrate or a pharmaceutically acceptable salt thereof.

According to this embodiment, moderate or severe hypertriglyceridemia can be treated.

In a preferred first embodiment, the therapeutically effective amount of pemafibrate or pharmaceutically acceptable salt thereof is from 0.2 to 1.0 mg, administered orally per day.

In a preferred first embodiment, the therapeutically effective amount of pemafibrate or pharmaceutically acceptable salt thereof is about 0.4 mg, administered orally per day.

In a preferred first embodiment, the therapeutically effective amount of pemafibrate or pharmaceutically acceptable salt thereof is administered twice daily.

In a preferred first embodiment, the patient has normal renal function.

In a preferred first embodiment, the patient has mild or moderate renal impairment.

In another preferred first embodiment the subject has an HDL-C concentration of less than 40 mg/dL.

In another preferred first embodiment the subject is on moderate or high intensity statin therapy.

In another preferred first embodiment the subject has an LDL-C concentration less than 70 mg/dL.

In another preferred first embodiment the subject has an HDL-C concentration of less than 40 mg/dL and is on moderate to high intensity statin therapy.

In another preferred first embodiment the subject or renally impaired patient has an HDL-C concentration of less than 40 mg/dL and has an LDL-C concentration less than 70 mg/dL.

In another preferred first embodiment the method prevents a cardiovascular event selected from nonfatal myocardial infarction, nonfatal ischemic stroke, hospitalization for unstable angina requiring unplanned coronary revascularization, cardiovascular death, and combinations thereof.

In another preferred first embodiment the subject has an HDL-C concentration of less than 40 mg/dL and is on moderate to high intensity statin therapy and has a triglyceride concentration of from 200 to 500 mg/dL.

In another preferred first embodiment the subject has an HDL-C concentration of less than 40 mg/dL and has an LDL-C concentration less than 70 mg/dL and has a triglyceride concentration of from 200 to 500 mg/dL.

In a second embodiment, the invention provides a method of treating severe hypertriglyceridemia in a subject in need thereof, comprising: (a) identifying a subject having a fasting baseline triglyceride level of about 500 mg/dl (5.65 mmol/L) and over, and (b) administering to the subject a pharmaceutical composition comprising pernafibrate or a pharmaceutically acceptable salt thereof.

In a preferred second embodiment, the therapeutically effective amount of pemafibrate or pharmaceutically acceptable salt thereof is from 0.2 to 1.0 mg, administered orally per day.

In a preferred second embodiment, the therapeutically effective amount of pemafibrate or pharmaceutically acceptable salt thereof is about 0.4 mg, administered orally per day.

In a preferred second embodiment, the therapeutically effective amount of pemafibrate or pharmaceutically acceptable salt thereof is administered twice daily.

In a preferred second embodiment, the patient has normal renal function.

In a preferred second embodiment, the patient has mild or moderate renal impairment.

In another preferred first embodiment the subject has an HDL-C concentration of less than 40 mg/dL.

In another preferred second embodiment the subject is on moderate or high intensity statin therapy.

In another preferred second embodiment the subject has an LDL-C concentration less than 70 mg/dL.

In another preferred second embodiment the subject has an HDL-C concentration of less than 40 mg/dL and is on moderate to high intensity statin therapy.

In another preferred second embodiment the subject or renally impaired patient has an HDL-C concentration of less than 40 mg/dL and has an LDL-C concentration less than 70 mg/dL.

In another preferred second embodiment the method prevents a cardiovascular event selected from nonfatal myocardial infarction, nonfatal ischemic stroke, hospitalization for unstable angina requiring unplanned coronary revascularization, cardiovascular death, and combinations thereof.

In a third embodiment, the invention provides a method of treating severe hypertriglyceridemia in a subject in need thereof, comprising: (a) identifying a subject having a fasting baseline triglyceride level of about 500 mg/dl (5.65 mmol/L) to about 2000 mg/dl (22.6 mmol/L), and (b) administering to the subject a pharmaceutical composition comprising pemafibrate or a pharmaceutically acceptable salt thereof.

In a preferred third embodiment, the therapeutically effective amount of pemafibrate or pharmaceutically acceptable salt thereof is from 0.2 to 1.0 mg, administered orally per day.

In a preferred third embodiment, the therapeutically effective amount of pemafibrate or pharmaceutically acceptable salt thereof is about 0.4 mg, administered orally per day.

In a preferred third embodiment, the therapeutically effective amount of pemafibrate or pharmaceutically acceptable salt thereof is administered twice daily.

In a preferred third embodiment, the patient has normal renal function.

In a preferred third embodiment, the patient has mild or moderate renal impairment.

In another preferred third embodiment the subject has an HDL-C concentration of less than 40 mg/dL.

In another preferred third embodiment the subject is on moderate or high intensity statin therapy.

In another preferred third embodiment the subject has an LDL-C concentration less than 70 mg/dL.

In another preferred third embodiment the subject has an HDL-C concentration of less than 40 mg/dL and is on moderate to high intensity statin therapy.

In another preferred third embodiment the subject or renally impaired patient has an HDL-C concentration of less than 40 mg/dL and has an LDL-C concentration less than 70 mg/dL.

In another preferred third embodiment the method prevents a cardiovascular event selected from nonfatal myocardial infarction, nonfatal ischemic stroke, hospitalization for unstable angina requiring unplanned coronary revascularization, cardiovascular death, and combinations thereof.

The dosing of the pemafibrate is preferably defined based on route of administration, dose, and length of treatment. The preferred route of administration is oral. Pemafibrate can be administered to a patient in the fed or fasting state.

The therapeutically effective amount of pemafibrate can be defined as a range of suitable doses on a daily basis. Thus, in one embodiment the therapeutically effective amount is from 0.1 to 1.0 mg of pemafibrate or a pharmaceutically acceptable salt thereof, administered orally per day. In another embodiment the therapeutically effective amount is from 0.2 to 0.8 mg of pemafibrate or a pharmaceutically acceptable salt thereof, administered orally per day. In still another embodiment the therapeutically effective amount is about 0.4 mg of pemafibrate or a pharmaceutically acceptable salt thereof, administered orally per day. Unless otherwise stated, these doses are based on the weight of the free base of pemafibrate.

The dose of pemafibrate can be administered as one dose per day or in two, three or four evenly divided doses per day.

In some embodiments, pemafibrate can be administered for a therapeutically effective period of time. The therapeutically effective period of time refers to the period of time necessary to treat moderate or severe hypertriglyceridemia, and varies depending on the conditions of a patient being treated and other factors such as the patient's age. The therapeutically effective period of time generally equates to three or more months of treatment, six or more months, one or more years, two or more years, three or more years, or four or more years.

Advantages of the invention are set forth in part in the foregoing description, and in part will be obvious from the description, or may be learned by practice of the invention. The advantages of the invention will be realized and attained by means of the elements and combinations particularly pointed out in the appended claims. It is to be understood that both the description herein is exemplary and explanatory only and not restrictive of the invention, as claimed.

Other embodiments of the invention may be apparent to those skilled in the art from consideration of the specification and practice of the invention disclosed herein. It is intended that the specification and examples be considered as exemplary only, with a true scope and spirit of the invention being indicated by the following claims.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated, and are intended to be purely exemplary of the invention and are not intended to limit the scope of what the inventors regard as their invention.

### Example 1 Treatment of Severe Hypertriglyceridemia with Pemafibrate

### Title of Study:

A Phase 3, Multi-Center, Placebo-Controlled, Randomized, Double-Blind, 12-Week Study With a 40-Week, Active-Controlled, Double-Blind Extension to Evaluate the Efficacy and Safety of Pemafibrate in Adult Patients With Fasting Triglyceride Levels≥500 mg/dL and <2000 mg/dL and Normal Renal Function.

### Study Design:

Study K-877-301 is a Phase 3, multi-center, randomized, double-blind study to confirm the efficacy and safety of pemafibrate 0.2 mg twice daily compared to matching placebo (in the 12-week Efficacy Period) and an active comparator, fenofibrate (in the 40-week Extension Period), in patients with fasting triglyceride (TG) levels≥500 mg/dL (5.65 mmol/L) and <2000 mg/dL (22.60 mmol/L) and normal renal function.

Eligible patients enter a 4- to 6-week lifestyle stabilization period (4-week stabilization for patients not requiring washout and 6-week washout and stabilization for patients on lipid-altering therapy other than statins, ezetimibe, or proprotein convertase subtilisin/kexin type 9 [PCSK9] inhibitors). The stabilization period is followed by a 2-week TG qualifying period (Visits 2 [Week −2] and 3 [Week −1]), and patient eligibility is assessed based on the mean TG value from these 2 visits. If the patient's mean TG level during the TG qualifying period is ≥450 mg/dL (5.09 mmol/L) and <500 mg/dL (5.65 mmol/L), an additional TG measurement can be taken 1 week later at Visit 3.1. The mean of all 3 TG measurements is used to determine eligibility for the study. After confirmation of qualifying fasting TG values, eligible patients enter a 12-week, randomized, double-blind Efficacy Period. At Visit 4 (Day 1), patients are randomly assigned in a 2:1 ratio to pemafibrate 0.2 mg twice daily or identical matching placebo tablets twice daily. During the 12-week Efficacy Period, patients return to the site at Visit 5 (Week 4), Visit 6 (Week 8), and Visit 7 (Week 12) for efficacy and safety evaluations.

Patients who successfully complete the 12-week Efficacy Period are eligible to continue in a 40-week, double-blind, active-controlled Extension Period after completing the Visit 7 (Week 12) procedures. Patients randomized to receive pemafibrate 0.2 mg twice daily in the 12-week Efficacy Period continue to receive pemafibrate 0.2 mg twice daily, as well as placebo matching fenofibrate 145 mg once daily, in the 40-week Extension Period. Patients randomized to receive placebo matching pemafibrate 0.2 mg twice daily in the 12-week Efficacy Period receive fenofibrate 145 mg once daily and placebo matching pemafibrate 0.2 mg twice daily in the 40-week Extension Period.

From Visit 7 (Week 12), patients not on statins, ezetimibe, or PCSK9 inhibitors may initiate therapy, and patients receiving statins, ezetimibe, or PCSK9 inhibitors may alter their dose, as indicated by guidelines or local standard of care.

After Visit 8 (Week 16), patients are to return to the site every 12 weeks until the last visit (Visit 11 [Week 52]).

### Primary Objective:

The primary objective of the study is to demonstrate the efficacy of pemafibrate 0.2 mg twice daily compared to placebo from baseline to Week 12 in lowering fasting TG levels in patients with fasting TG levels≥500 mg/dL (5.65 mmol/L) and <2000 mg/dL (22.60 mmol/L).

### Secondary Objectives:

The secondary objectives of the study are the following:
To evaluate the efficacy of pemafibrate 0.2 mg twice daily from baseline to Week 52 in lowering fasting TG levels in patients with fasting TG levels≥500 mg/dL (5.65 mmol/L) and <2000 mg/dL (22.60 mmol/L);
To evaluate the efficacy of pemafibrate 0.2 mg twice daily from baseline to Week 12 and Week 52 in altering lipid parameters in patients with fasting TG levels≥500 mg/dL (5.65 mmol/L) and <2000 mg/dL (22.60 mmol/L);
To evaluate the safety and tolerability of pemafibrate 0.2 mg twice daily in patients with fasting TG levels≥500 mg/dL (5.65 mmol/L) and <2000 mg/dL (22.60 mmol/L); and
To determine the plasma concentrations of pemafibrate for the purpose of use in population pharmacokinetic (PK) analysis and PK/pharmacodynamic (PD) analysis.

### Exploratory Objective:

The exploratory objective of the study is to evaluate signs of potential efficacy of pemafibrate 0.2 mg twice daily in treating non-alcoholic fatty liver disease in patients with fasting TG levels≥500 mg/dL (5.65 mmol/L) and <2000 mg/dL (22.60 mmol/L)

### Patient Population:

The study population consist of male and female patients 218 years of age with fasting TG levels≥500 mg/dL (5.65 mmol/L) and <2000 mg/dL (22.60 mmol/L) after washout from background lipid-altering therapy other than statins, ezetimibe, or PCSK9 inhibitors and with normal renal function. Stable therapy with statins, ezetimibe, or PCSK9 inhibitors is allowed. The 40-week, active-controlled Extension Period population consists of patients completing the 12-week placebo-controlled Efficacy Period. Patients in the 40-week, active-controlled Extension Period are allowed to continue in the study even if the background lipid-altering therapy with statins, ezetimibe, or PCSK9 inhibitors requires adjustment.

### Number of Patients:

Approximately 630 patients (420 patients receiving pemafibrate; 210 patients receiving placebo/fenofibrate)

### Dose Levels:

### 12-Week Efficacy Period

Pemafibrate: 0.2 mg twice daily

Placebo: twice daily

### 40-Week Extension Period

Pemafibrate: 0.2 mg twice daily/fenofibrate matching placebo: once daily

Fenofibrate: 145 mg once daily/Pemafibrate matching placebo: twice daily

### Route of Administration:

### Oral

### Duration of Treatment:

This study consists of a 12-week, double-blind, placebo-controlled Efficacy Period, followed by a 40-week, double-blind, active-controlled Extension Period, for a total of 52 weeks on study drug.

### Criteria for Evaluation:

### Efficacy:

The primary efficacy endpoint is the percent change in fasting TG from baseline to Week 12. Baseline for TG is defined as the mean of Visit 4 (Day 1) and the preceding TG qualifying visit (either Visit 3 [Week −1] or Visit 3.1, if required) measurements.

The secondary efficacy endpoints for the 12-week Efficacy Period include the following:
Percent change from baseline to Week 12 in remnant cholesterol (calculated as total cholesterol [TC]−low-density lipoprotein C [LDL-C]−high-density lipoprotein C [HDL-C]), HDL-C, apolipoprotein (Apo) A1, and non-HDL-C;
   Low-density lipoprotein cholesterol is determined by preparative ultracentrifugation;
Percent change from baseline to Week 12 in TC, LDL-C, free fatty acids (FFAs), Apo A2, Apo B, Apo B48, Apo B100, Apo C2, Apo C3, and Apo E;
Change from baseline to Week 12 in fibroblast growth factor 21 (FGF21) and high-sensitivity C-reactive protein (hsCRP), and percent change from baseline to Week 12 in ion mobility analysis and lipoprotein fraction (nuclear magnetic resonance [NMR]); and
Percent change from baseline to Week 12 in the lipid and lipoprotein ratios of TG:HDL-C, TC:HDL-C, non-HDL-C:HDL-C, LDL-C:Apo B, Apo B:Apo A1, and Apo C3:Apo C2.
   The secondary efficacy endpoints for the 40-week Extension Period include the following:
Percent change from baseline to Week 52 in fasting TG;
Percent change from baseline to Week 52 in remnant cholesterol (calculated as TC−LDL-C−HDL-C), HDL-C, Apo A1, and non-HDL-C;
   Low-density lipoprotein cholesterol is determined by preparative ultracentrifugation;
Percent change from baseline to Week 52 in TC, LDL-C, FFAs, Apo A2, Apo B, Apo B48, Apo B100, Apo C2, Apo C3, and Apo E;
Change from baseline to Week 52 in FGF21 and hsCRP, and percent change from baseline to Week 52 in ion mobility analysis and lipoprotein fraction (NMR); and
Percent change from baseline to Week 52 in the lipid and lipoprotein ratios of TG:HDL-C, TC:HDL-C, non-HDL-C:HDL-C, LDL-C:Apo B, Apo B:Apo A1, and Apo C3:Apo C2.

Baseline for TG, TC, HDL-C, non-HDL-C, LDL-C, and remnant cholesterol are defined as the mean of Visit 4 (Day 1) and the preceding TG qualifying visit (either Visit 3 [Week −1] or Visit 3.1, if required) measurements. Baseline for all other efficacy and safety variables are defined as Visit 4 (Day 1). If the measurement at this visit is missing, the last measurement prior to the first dose of randomized study drug is used.

For patients randomized to receive placebo (in the 12-week Efficacy Period) and fenofibrate 145 mg (in the 40-week Extension Period), change from Visit 7 (Week 12) in efficacy and safety variables also are explored.

The exploratory efficacy endpoints for the 12-week Efficacy Period include the following:

Change from baseline to Week 12 in selected biomarkers suggestive of hepatic inflammation and fibrosis, including cytokeratin-18 (CK-18), ferritin, hyaluronic acid, tumor necrosis factor alpha (TNF-α), type IV collagen, and adiponectin.

The exploratory efficacy endpoints for the 40-week Extension Period include the following:

Change from baseline to Week 52 in selected biomarkers suggestive of hepatic inflammation and fibrosis, including CK-18, ferritin, hyaluronic acid, TNF-α, type IV collagen, and adiponectin.

### Safety:

Safety assessments include adverse events (AEs), clinical laboratory measurements (chemistry, hematology, coagulation profile, endocrinology, and urinalysis), 12-lead electrocardiograms (ECGs), vital signs (heart rate, respiratory rate, and blood pressure), and physical examinations.

### Pharmacokinetics/Pharmacodynamics:

Pharmacokinetic concentrations collected during the 12-week Efficacy Period is used for population PK analysis and PK/PD analysis.

### Inclusion Criteria:

Able to understand and willing to comply with all study requirements and procedures throughout the duration of the study and give written informed consent;

Aged≥18 years;

Patients receiving statin therapy must meet one of the following criteria:
Aged≥21 years with clinical atherosclerotic cardiovascular disease (ASCVD) (history of acute coronary syndrome or myocardial infarction, stable or unstable angina, coronary revascularization, stroke, transient ischemic attack [TIA] presumed to be of atherosclerotic origin, or peripheral arterial disease or revascularization), on a high intensity statin (or moderate intensity statin if not a candidate for high intensity statin due to safety concerns);
Aged≥21 years with a history of LDLC≥190 mg/dL, which is not due to secondary modifiable causes, on a high intensity statin (or moderate intensity statin if not a candidate for high intensity statin due to safety concerns);
Aged 40 to 75 years, inclusive, without clinical ASCVD but with diabetes and a history of LDLC of 70 to 189 mg/dL, inclusive, on a moderate or high intensity statin; or Aged 40 to 75 years, inclusive, without clinical ASCVD or diabetes, with a history of LDLC of 70 to 189 mg/dL, inclusive, with estimated 10 year risk for ASCVD of ≥7.5% by the Pooled Cohort Equation on a moderate or high intensity statin; Patients not currently on statins, must not meet the criteria for statin therapy listed above;

### Exclusion Criteria:

Patients who require lipid altering treatments other than study drugs (pemafibrate or fenofibrate), statins, ezetimibe, or PCSK9 inhibitors during the course of the study. These include bile acid sequestrants, non-study fibrates, niacin (>100 mg/day), omega 3 fatty acids (>1000 mg/day), or any supplements used to alter lipid metabolism including, but not limited to, red rice yeast supplements, garlic supplements, soy isoflavone supplements, sterol/stanol products, or policosanols;

Body mass index (BMI)>45 kg/m2 at Visit 1 (Week 8 or Week 6);

Patients with type 1 diabetes mellitus;

Patients with newly diagnosed (within 3 months prior to Visit 2 [Week 2])

or poorly controlled type 2 diabetes mellitus (T2DM), defined as hemoglobin A1c>9.5% at Visit 1 (Week 8 or Week 6);

### Statistical Analysis:

### Efficacy:

In order to control the family-wise Type I error at a 0.05 level, a fixed sequential testing procedure is implemented. In a hierarchical step-down manner, the primary endpoint is tested first, followed by secondary endpoints, tested in the following hierarchical manner: percent change from baseline to Week 12 in a fixed sequence of (1) remnant cholesterol (calculated as TC−LDL-C−HDL-C), (2) HDL-C, (3) Apo A1, and (4) non-HDL-C. Each test is planned to be performed at a 0.05 significance level. Inferential conclusions about these efficacy endpoints require statistical significance of the previous one.

For other efficacy endpoints, nominal p-values and 95% confidence intervals (CIs) is presented, but should not be considered as confirmatory.

The primary efficacy analysis is based on Hodges-Lehmann estimator with pattern-mixture model imputation based on the Full Analysis Set (FAS). The pattern-mixture model is used as the primary imputation method as part of the primary analysis for the percent change in fasting TGs from baseline to Week 12. This imputation model includes factors such as patient demographics, disease status, and baseline TG, as well as adherence to therapy. The imputation model imputes missing Week 12 TG values as follows:
For patients who do not adhere to therapy and who do not have a Week 12 measurement, the missing data imputation method use patients in the same treatment arm who do not adhere to therapy and have a Week 12 measurement; and
If there are no patients in the same treatment arm who do not adhere to therapy and have a Week 12 measurement, missing Week 12 TG values are imputed as follows:
   For the pemafibrate arm, the treatment effect is considered washed out and baseline TG values are used to impute the Week 12 TG values; and
   For the placebo arm, missing Week 12 TG values are imputed assuming missing at random, including patient demographics, disease status, and baseline and post-baseline efficacy data from the placebo arm.

After the multiple imputation step, each imputed dataset is analyzed by the nonparametric Hodges-Lehmann method and the Hodges-Lehmann estimator and standard error are combined to produce treatment difference estimate and 95% CI and p-value.

Other sensitivity methods are to be explored including (1) Hodges-Lehmann estimator with imputation method probabilities of missing estimated using logistic regression based on the FAS and (2) analysis of covariance (ANCOVA) of rank-transformed Week 12 percent change from baseline in TG with pattern-mixture model imputation based on the FAS. Additional statistical methods might be explored, including mixed effect model repeat measurement (MMRM) with percent change in TG from baseline based on the FAS.

The primary efficacy analysis is repeated on the Per-Protocol Set.

Summary statistics (number of patients, mean, standard deviation, median, minimum, maximum, 25^{th} percentile, and 75^{th} percentile) at baseline, each scheduled visit, and change and percent change in fasting TG from baseline to each scheduled visit is provided.

Secondary efficacy endpoints included in the hierarchical step-down testing procedure include percent change from baseline to Week 12 in a fixed sequence of (1) remnant cholesterol, (2) HDL-C, (3) Apo A1, and (4) non-HDL-C.

The secondary and exploratory efficacy endpoints during the 12-week Efficacy Period is analyzed using an ANCOVA model with the same imputation method used for the primary analysis. The ANCOVA model includes country, current statin therapy use (not on statin therapy versus currently receiving statin therapy), and treatment as factors; baseline value as a covariate. If the normality assumption is not met, the Hodges-Lehmann estimator with the same imputation method used for the primary analysis is used.

The secondary efficacy endpoint of percent change in fasting TG from baseline to Week 52 is summarized descriptively. Change from Visit 7 (Week 12) for the fenofibrate group during the 40-week Extension Period also is summarized for each visit. Other efficacy endpoints during the 40-week Extension Period will be summarized descriptively. No hypothesis testing is performed.

Analyses of selected primary, secondary, and exploratory efficacy variables may be performed for subgroups of patients based on statin therapy (statin therapy versus no statin therapy), gender, age (<65 years versus 265 years), race (white versus not white), ethnicity, and other baseline characteristics.

### Safety:

The safety endpoint data are summarized for the Safety Analysis Set for the 12-week Efficacy Period, 40-week Extension Period, and overall.

The AEs are coded using the latest version of the Medical Dictionary for Regulatory Activities. A general summary of the AEs and serious AEs (SAEs) are summarized by overall number of AEs, severity, and relationship to study drug per treatment group. The number of AEs leading to withdrawal and SAEs leading to death also are summarized. The incidence of AEs is summarized by body system and treatment group. The incidence of treatment-emergent AEs also is summarized by system organ class and preferred term.

The safety laboratory data are summarized by visit and by treatment group, along with changes from the baseline. The values that are below the lower limit or above the upper limit of the reference range are flagged. Those values or changes in values that are identified as being clinically significant are flagged. Laboratory abnormalities of special interest, such as liver function tests and pancreatitis events, are summarized.

Vital signs and 12-lead ECGs also are summarized by visit and by treatment group, along with the changes from baseline.

### Pharmacokinetics:

Population PK and PK/PD data are analyzed and reported separately. The concentration-time data are modeled using a population approach with compartment models, and the effects of patient characteristics are examined to determine if they influence drug exposure. Patient characteristics are include age, gender, ethnicity, BMI, country, etc. In addition, the relationship between drug concentration and safety variables are investigated. Safety variables include, but are not limited to, AST, ALT, alkaline phosphatase, and CK. Measures of exposure (predicted clearance, area under the concentration-time curve [AUC], and/or maximum plasma PK concentration [Cₘₐₓ]) are correlated with safety variables.

### Example 2 Treatment of Severe Hypertriglyceridemia with Pemafibrate

### Title of Study:

A Phase 3, Multi-Center, Placebo-Controlled, Randomized, Double-Blind, 12-Week Study With a 40-Week, Active-Controlled, Double-Blind Extension to Evaluate the Efficacy and Safety of Pemafibrate in Adult Patients With Fasting Triglyceride Levels≥500 mg/dL and <2000 mg/dL and Mild or Moderate Renal Impairment.

### Study Design:

Study K-877-303 is a Phase 3, multi-center, randomized, study to confirm the efficacy and safety of pemafibrate 0.2 mg twice daily compared to matching placebo (in the double-blind 12-week Efficacy Period) and an active comparator, fenofibrate (in the open-label 40-week Extension Period), in patients with fasting triglyceride (TG) levels≥500 mg/dL (5.65 mmol/L) and <2000 mg/dL (22.60 mmol/L) and mild or moderate renal impairment (estimated glomerular filtration rate [eGFR]≥30 mL/min/1.73 m² and <90 mL/min/1.73 m²).

Eligible patients enter a 4- to 6-week lifestyle stabilization period (4-week stabilization for patients not requiring washout and 6-week washout and stabilization for patients on lipid-altering therapy other than statins, ezetimibe, or proprotein convertase subtilisin/kexin type 9 [PCSK9] inhibitors). The stabilization period is followed by a 2-week TG qualifying period (Visits 2 [Week −2] and 3 [Week −1]), and patient eligibility is assessed based on the mean TG value from these 2 visits. If the patient's mean TG level during the TG qualifying period is ≥450 mg/dL (5.09 mmol/L) and <500 mg/dL (5.65 mmol/L), an additional TG measurement can be taken 1 week later at Visit 3.1. The mean of all 3 TG measurements is used to determine eligibility for the study. After confirmation of qualifying fasting TG values, eligible patients enter a 12-week, randomized, double-blind Efficacy Period. At Visit 4 (Day 1), patients are randomly assigned in a 2:1 ratio to pemafibrate 0.2 mg twice daily or identical matching placebo tablets twice daily. During the 12-week Efficacy Period, patients return to the site at Visit 5 (Week 4), Visit 6 (Week 8), and Visit 7 (Week 12) for efficacy and safety evaluations.

Patients who successfully complete the 12-week Efficacy Period are eligible to continue in a 40-week, open-label, active-controlled Extension Period after completing the Visit 7 (Week 12) procedures. Patients randomized to receive pemafibrate 0.2 mg twice daily in the 12-week Efficacy Period continue to receive pemafibrate 0.2 mg twice daily in the 40-week Extension Period. Patients randomized to receive placebo matching pemafibrate 0.2 mg twice daily in the 12-week Efficacy Period initiate fenofibrate dosing at 48 mg once daily at Visit 7 (Week 12). Starting from Visit 8 (Week 16), Investigators can adjust fenofibrate dosing (to 145 mg once daily) at their discretion according to the local standard of care.

From Visit 7 (Week 12), patients not on statins, ezetimibe, or PCSK9 inhibitors may initiate therapy, and patients receiving statins, ezetimibe, or PCSK9 inhibitors may alter their dose, as indicated by guidelines or local standard of care.

After Visit 8 (Week 16), patients are to return to the site every 12 weeks until the last visit (Visit 11 [Week 52]).

### Primary Objective:

The primary objective of the study is to demonstrate the efficacy of pemafibrate 0.2 mg twice daily compared to placebo from baseline to Week 12 in lowering fasting TG levels in patients with fasting TG levels≥500 mg/dL (5.65 mmol/L) and <2000 mg/dL (22.60 mmol/L) and mild or moderate renal impairment.

### Secondary Objectives:

The secondary objectives of the study are the following:
To evaluate the efficacy of pemafibrate 0.2 mg twice daily from baseline to Week 52 in lowering fasting TG levels in patients with fasting TG levels≥500 mg/dL (5.65 mmol/L) and <2000 mg/dL (22.60 mmol/L) and mild or moderate renal impairment;
To evaluate the efficacy of pemafibrate 0.2 mg twice daily from baseline to Week 12 and Week 52 in altering lipid parameters in patients with fasting TG levels≥500 mg/dL (5.65 mmol/L) and <2000 mg/dL (22.60 mmol/L) and mild or moderate renal impairment;
To evaluate the safety and tolerability of pemafibrate 0.2 mg twice daily in patients with fasting TG levels≥500 mg/dL (5.65 mmol/L) and <2000 mg/dL (22.60 mmol/L) and mild or moderate renal impairment; and
To determine the plasma concentrations of pemafibrate for the purpose of use in population pharmacokinetic (PK) analysis and PK/pharmacodynamic (PD) analysis.

### Exploratory Objective:

The exploratory objective of the study is to evaluate signs of potential efficacy of pemafibrate 0.2 mg twice daily in treating non-alcoholic fatty liver disease in patients with fasting TG levels≥500 mg/dL (5.65 mmol/L) and <2000 mg/dL (22.60 mmol/L) and mild or moderate renal impairment.

### Patient Population:

The study population consist of male and female patients 218 years of age with fasting TG levels≥500 mg/dL (5.65 mmol/L) and <2000 mg/dL (22.60 mmol/L) after washout from background lipid-altering therapy other than statins, ezetimibe, or PCSK9 inhibitors and with normal renal function. Stable therapy with statins, ezetimibe, or PCSK9 inhibitors is allowed. The 40-week, active-controlled Extension Period population consists of patients completing the 12-week placebo-controlled Efficacy Period. Patients in the 40-week, active-controlled Extension Period are allowed to continue in the study even if the background lipid-altering therapy with statins, ezetimibe, or PCSK9 inhibitors requires adjustment.

### Number of Patients:

Approximately 420 patients (280 patients receiving pemafibrate; 140 patients receiving placebo/fenofibrate)

### Dose Levels:

### 12-Week Efficacy Period

Pemafibrate: 0.2 mg twice daily

Placebo: twice daily

### 40-Week Extension Period

Pemafibrate: 0.2 mg twice daily/fenofibrate matching placebo: once daily

Fenofibrate: 48 mg once daily or 145 mg once daily/Pemafibrate matching placebo: twice daily

### Route of Administration:

### Oral

### Duration of Treatment:

This study consists of a 12-week, double-blind, placebo-controlled Efficacy Period, followed by a 40-week, double-blind, active-controlled Extension Period, for a total of 52 weeks on study drug.

### Criteria for Evaluation:

### Efficacy:

The primary efficacy endpoint is the percent change in fasting TG from baseline to Week 12. Baseline for TG is defined as the mean of Visit 4 (Day 1) and the preceding TG qualifying visit (either Visit 3 [Week −1] or Visit 3.1, if required) measurements.

The secondary efficacy endpoints for the 12-week Efficacy Period include the following:
Percent change from baseline to Week 12 in remnant cholesterol (calculated as total cholesterol [TC]−low-density lipoprotein C [LDL-C]−high-density lipoprotein C [HDL-C]), HDL-C, apolipoprotein (Apo) A1, and non-HDL-C;
   Low-density lipoprotein cholesterol is determined by preparative ultracentrifugation;
Percent change from baseline to Week 12 in TC, LDL-C, free fatty acids (FFAs), Apo A2, Apo B, Apo B48, Apo B100, Apo C2, Apo C3, and Apo E;
Change from baseline to Week 12 in fibroblast growth factor 21 (FGF21) and high-sensitivity C-reactive protein (hsCRP), and percent change from baseline to Week 12 in ion mobility analysis and lipoprotein fraction (nuclear magnetic resonance [NMR]); and
Percent change from baseline to Week 12 in the lipid and lipoprotein ratios of TG: HDL-C, TC: HDL-C, non-HDL-C:HDL-C, LDL-C:Apo B, Apo B:Apo A1, and Apo C3:Apo C2.
   The secondary efficacy endpoints for the 40-week Extension Period include the following:
Percent change from baseline to Week 52 in fasting TG;
Percent change from baseline to Week 52 in remnant cholesterol (calculated as TC−LDL-C−HDL-C), HDL-C, Apo A1, and non-HDL-C;
   Low-density lipoprotein cholesterol is determined by preparative ultracentrifugation;
Percent change from baseline to Week 52 in TC, LDL-C, FFAs, Apo A2, Apo B, Apo B48, Apo B100, Apo C2, Apo C3, and Apo E;
Change from baseline to Week 52 in FGF21 and hsCRP, and percent change from baseline to Week 52 in ion mobility analysis and lipoprotein fraction (NMR); and
Percent change from baseline to Week 52 in the lipid and lipoprotein ratios of TG:HDL-C, TC:HDL-C, non-HDL-C:HDL-C, LDL-C:Apo B, Apo B:Apo A1, and Apo C3:Apo C2.

Baseline for TG, TC, HDL-C, non-HDL-C, LDL-C, and remnant cholesterol are defined as the mean of Visit 4 (Day 1) and the preceding TG qualifying visit (either Visit 3 [Week −1] or Visit 3.1, if required) measurements. Baseline for all other efficacy and safety variables are defined as Visit 4 (Day 1). If the measurement at this visit is missing, the last measurement prior to the first dose of randomized study drug is used.

For patients randomized to receive placebo (in the 12-week Efficacy Period) and fenofibrate (in the 40-week Extension Period), change from Visit 7 (Week 12) in efficacy and safety variables also are explored.

The exploratory efficacy endpoints for the 12-week Efficacy Period include the following:

Change from baseline to Week 12 in selected biomarkers suggestive of hepatic inflammation and fibrosis, including cytokeratin-18 (CK-18), ferritin, hyaluronic acid, tumor necrosis factor alpha (TNF-α), type IV collagen, and adiponectin.

The exploratory efficacy endpoints for the 40-week Extension Period include the following:

Change from baseline to Week 52 in selected biomarkers suggestive of hepatic inflammation and fibrosis, including CK-18, ferritin, hyaluronic acid, TNF-α, type IV collagen, and adiponectin.

### Safety:

Safety assessments include adverse events (AEs), clinical laboratory measurements (chemistry, hematology, coagulation profile, endocrinology, and urinalysis), 12-lead electrocardiograms (ECGs), vital signs (heart rate, respiratory rate, and blood pressure), and physical examinations.

### Pharmacokinetics/Pharmacodynamics:

Pharmacokinetic concentrations collected during the 12-week Efficacy Period is used for population PK analysis and PK/PD analysis.

### Inclusion Criteria:

Able to understand and willing to comply with all study requirements and procedures throughout the duration of the study and give written informed consent;

Aged≥18 years;

Patients receiving statin therapy must meet one of the following criteria:
Aged≥21 years with clinical atherosclerotic cardiovascular disease (ASCVD) (history of acute coronary syndrome or myocardial infarction, stable or unstable angina, coronary revascularization, stroke, transient ischemic attack [TIA] presumed to be of atherosclerotic origin, or peripheral arterial disease or revascularization), on a highintensity statin (or moderate intensity statin if not a candidate for high intensity statin due to safety concerns);
Aged≥21 years with a history of LDLC≥190 mg/dL, which is not due to secondary modifiable causes, on a high intensity statin (or moderate intensity statin if not a candidate for high intensity statin due to safety concerns);
Aged 40 to 75 years, inclusive, without clinical ASCVD but with diabetes and a history of LDLC of 70 to 189 mg/dL, inclusive, on a moderate or high intensity statin; or
Aged 40 to 75 years, inclusive, without clinical ASCVD or diabetes, with a history of LDLC of 70 to 189 mg/dL, inclusive, with estimated 10 year risk for ASCVD of ≥7.5% by the Pooled Cohort Equation on a moderate or high intensity statin;
Patients not currently on statins, must not meet the criteria for statin therapy listed above;
Fasting TG levels≥500 mg/dL (5.65 mmol/L) and <2000 mg/dL (22.60 mmol/L) based on the mean of Visit 2 (Week −2) and Visit 3 (Week −1);
Normal renal function (i.e., estimated glomerular filtration rate [eGFR]≥90 mL/min/1.73 m2) at Visit 1 (Week −8 or Week −6);

### Exclusion Criteria:

Patients who require lipid altering treatments other than study drugs (pemafibrate or fenofibrate), statins, ezetimibe, or PCSK9 inhibitors during the course of the study. These include bile acid sequestrants, non-study fibrates, niacin (>100 mg/day), omega 3 fatty acids (>1000 mg/day), or any supplements used to alter lipid metabolism including, but not limited to, red rice yeast supplements, garlic supplements, soy isoflavone supplements, sterol/stanol products, or policosanols;

Body mass index (BMI)>45 kg/m2 at Visit 1 (Week 8 or Week 6);

Patients with type 1 diabetes mellitus;

Patients with newly diagnosed (within 3 months prior to Visit 2 [Week 2]) or poorly controlled type 2 diabetes mellitus (T2DM), defined as hemoglobin A1c>9.5% at Visit 1 (Week 8 or Week 6);

### Statistical Analysis:

### Efficacy:

In order to control the family-wise Type I error at a 0.05 level, a fixed sequential testing procedure is implemented. In a hierarchical step-down manner, the primary endpoint is tested first, followed by secondary endpoints, tested in the following hierarchical manner: percent change from baseline to Week 12 in a fixed sequence of (1) remnant cholesterol (calculated as TC−LDL-C−HDL-C), (2) HDL-C, (3) Apo A1, and (4) non-HDL-C. Each test is planned to be performed at a 0.05 significance level. Inferential conclusions about these efficacy endpoints require statistical significance of the previous one.

For other efficacy endpoints, nominal p-values and 95% confidence intervals (CIs) is presented, but should not be considered as confirmatory.

The primary efficacy analysis is based on Hodges-Lehmann estimator with pattern-mixture model imputation based on the Full Analysis Set (FAS). The pattern-mixture model is used as the primary imputation method as part of the primary analysis for the percent change in fasting TGs from baseline to Week 12. This imputation model includes factors such as patient demographics, disease status, and baseline TG, as well as adherence to therapy. The imputation model imputes missing Week 12 TG values as follows:
For patients who do not adhere to therapy and who do not have a Week 12 measurement, the missing data imputation method use patients in the same treatment arm who do not adhere to therapy and have a Week 12 measurement; and
If there are no patients in the same treatment arm who do not adhere to therapy and have a Week 12 measurement, missing Week 12 TG values are imputed as follows:
   For the pemafibrate arm, the treatment effect is considered washed out and baseline TG values are used to impute the Week 12 TG values; and
   For the placebo arm, missing Week 12 TG values are imputed assuming missing at random, including patient demographics, disease status, and baseline and post-baseline efficacy data from the placebo arm.

After the multiple imputation step, each imputed dataset is analyzed by the nonparametric Hodges-Lehmann method and the Hodges-Lehmann estimator and standard error are combined to produce treatment difference estimate and 95% CI and p-value.

Other sensitivity methods are to be explored including (1) Hodges-Lehmann estimator with imputation method probabilities of missing estimated using logistic regression based on the FAS and (2) analysis of covariance (ANCOVA) of rank-transformed Week 12 percent change from baseline in TG with pattern-mixture model imputation based on the FAS. Additional statistical methods might be explored, including mixed effect model repeat measurement (MMRM) with percent change in TG from baseline based on the FAS.

The primary efficacy analysis is repeated on the Per-Protocol Set.

Summary statistics (number of patients, mean, standard deviation, median, minimum, maximum, 25^{th} percentile, and 75^{th} percentile) at baseline, each scheduled visit, and change and percent change in fasting TG from baseline to each scheduled visit is provided.

Secondary efficacy endpoints included in the hierarchical step-down testing procedure include percent change from baseline to Week 12 in a fixed sequence of (1) remnant cholesterol, (2) HDL-C, (3) Apo A1, and (4) non-HDL-C.

The secondary and exploratory efficacy endpoints during the 12-week Efficacy Period is analyzed using an ANCOVA model with the same imputation method used for the primary analysis. The ANCOVA model includes country, current statin therapy use (not on statin therapy versus currently receiving statin therapy), and treatment as factors; baseline value as a covariate. If the normality assumption is not met, the Hodges-Lehmann estimator with the same imputation method used for the primary analysis is used.

The secondary efficacy endpoint of percent change in fasting TG from baseline to Week 52 is summarized descriptively. Change from Visit 7 (Week 12) for the fenofibrate group during the 40-week Extension Period also is summarized for each visit. Other efficacy endpoints during the 40-week Extension Period will be summarized descriptively. No hypothesis testing is performed.

Analyses of selected primary, secondary, and exploratory efficacy variables may be performed for subgroups of patients based on statin therapy (statin therapy versus no statin therapy), gender, age (<65 years versus≥65 years), race (white versus not white), ethnicity, and other baseline characteristics.

### Safety:

The safety endpoint data are summarized for the Safety Analysis Set for the 12-week Efficacy Period, 40-week Extension Period, and overall.

The AEs are coded using the latest version of the Medical Dictionary for Regulatory Activities. A general summary of the AEs and serious AEs (SAEs) are summarized by overall number of AEs, severity, and relationship to study drug per treatment group. The number of AEs leading to withdrawal and SAEs leading to death also are summarized. The incidence of AEs is summarized by body system and treatment group. The incidence of treatment-emergent AEs also is summarized by system organ class and preferred term.

The safety laboratory data are summarized by visit and by treatment group, along with changes from the baseline. The values that are below the lower limit or above the upper limit of the reference range are flagged. Those values or changes in values that are identified as being clinically significant are flagged. Laboratory abnormalities of special interest, such as liver function tests and pancreatitis events, are summarized.

Vital signs and 12-lead ECGs also are summarized by visit and by treatment group, along with the changes from baseline.

### Pharmacokinetics:

Population PK and PK/PD data are analyzed and reported separately. The concentration-time data are modeled using a population approach with compartment models, and the effects of patient characteristics are examined to determine if they influence drug exposure. Patient characteristics are include age, gender, ethnicity, BMI, country, etc. In addition, the relationship between drug concentration and safety variables are investigated. Safety variables include, but are not limited to, AST, ALT, alkaline phosphatase, and CK. Measures of exposure (predicted clearance, area under the concentration-time curve [AUC], and/or maximum plasma PK concentration [Cₘₐₓ]) are correlated with safety variables.

### REFERENCES CITED

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.
1 Miller M, Stone N J, Ballantyne C, et al. American Heart Association Clinical Lipidology, Thrombosis, and Prevention Committee of the Council on Nutrition, Physical Activity, and Metabolism; Council on Arteriosclerosis, Thrombosis and Vascular Biology; Council on Cardiovascular Nursing; Council on the Kidney in Cardiovascular Disease. Triglycerides and cardiovascular disease: a scientific statement from the American Heart Association. Circulation. 2011; 123(20):2292-2333.
2 Scherer J, Singh V P, Pitchumoni C S, Yadav D. Issues in hypertriglyceridemic pancreatitis: an update. J Clin Gastroenterol. 2014; 48(3):195-203.
3 Anderson F, Thomson S R, Clarke D L, Buccimazza I. Dyslipidaemic pancreatitis clinical assessment and analysis of disease severity and outcomes. Pancreatology. 2009; 9(3):252-257.
4 Deng L H, Xue P, Xia Q, Yang N X, Wan M H. Effect of admission hypertriglyceridemia on the episodes of severe acute pancreatitis. World J Gastroenterol. 2008; 14(28):4558-4561.
5 Staels B, Dallongeville J, Auwerx J, Schoonjans K, Leitersdorf E, Fruchart J C. Mechanism of action of fibrates on lipid and lipoprotein metabolism. Circulation. 1998; 98:2088-2093.
6 National Institutes of Health, National Heart, Lung, and Blood Institute. Third Report of the National Cholesterol Education Program (NCEP) Expert Panel on Detection, Evaluation, and Treatment of High Blood Cholesterol in Adults (Adult Treatment Panel III) Final Report. 2002; pII-1-61; September 2002.
7 Pirillo A, Catapano A L. Update on the management of severe hypertriglyceridemia—focus on free fatty acid forms of omega-3. Drug Des Devel Ther. 2015; 9:2128-2137.
8 Task Force for the management of dyslipidaemias of the European Society of Cardiology (ESC) and the European Atherosclerosis Society (EAS). ESC/EAS Guidelines for the management of dyslipidaemias. Eur Heart J. 2011; 32:1769-1818.
9 Package insert for Lipidil® Tablets, revised May 2012 (2nd edition).
10 Package insert for Bezatol® S R Tablets, revised June 2009 (12th edition).
11 Package insert for Clofibrate Capsules, revised July 2009 (6th edition).
12 Ciprofibrate 100 mg tablets, Summary of Product Characteristics at eMedicines Compendium.
13 Lopid 300 mg capsules and 600 mg tablets, Summary of Product Characteristics at eMedicines Compendium.
14 Package ins ert for Tricor® tablets, revised September 2011.
15 Package insert for Trilipix® capsule, revised September 2012.
16 Package insert for Lopid® tablets, revised September 2010.

1) A method of treating moderate or severe hypertriglyceridemia in a subject in need thereof, comprising administering to the patient a therapeutically effective amount of pemafibrate or a pharmaceutically acceptable salt thereof.

2) A method of treating severe hypertriglyceridemia in a subject in need thereof, comprising: (a) identifying a subject having a fasting baseline triglyceride level of about 500 mg/dl (5.65 mmol/L) and over, and (b) administering to the subject a pharmaceutical composition comprising pernafibrate or a pharmaceutically acceptable salt thereof.

3) The method according to claim 1, wherein the therapeutically effective amount of pemafibrate or pharmaceutically acceptable salt thereof is from 0.2 to 1.0 mg, administered orally per day.

4) The method according to claim 1, wherein the therapeutically effective amount of pemafibrate or pharmaceutically acceptable salt thereof is 0.4 mg, administered orally per day.

5) The method according to claim 1, wherein the patient has mild or moderate renal impairment.

6) The method according to claim 1, wherein the patient has severe renal impairment.

7) The method of claim 1, wherein the patient is on high intensity statin therapy and aged≥21 years with clinical ASCVD selected from a history of acute coronary syndrome or myocardial infarction, stable or unstable angina, coronary revascularization, stroke, transient ischemic attack [TIA] of atherosclerotic origin, or peripheral arterial disease or revascularization.

8) The method of claim 1, wherein the patient is on high intensity statin therapy and aged≥21 years with a history of LDL-C≥190 mg/dL, which is not due to secondary modifiable causes.

9) The method of claim 1, wherein the patient is on moderate or high intensity statin therapy and aged 40 to 75 years, inclusive, without clinical ASCVD but with type-2 diabetes and a history of LDL-C of 70 to 189 mg/dL, inclusive.

10) The method of claim 1, wherein the patient is on moderate or high intensity statin therapy and aged 40 to 75 years, inclusive, without clinical ASCVD or diabetes, with a history of LDL-C of 70 to 189 mg/dL, inclusive, with estimated 10-year risk for ASCVD of ≥7.5% by the Pooled Cohort Equation.

11) The method of claim 1 wherein the subject has one or a combination of low HDL-C levels, elevated LDL-C levels, elevated non-HDL-C levels, or elevated Total Cholesterol levels.

12) The method of claim 1, wherein the subject has a fasting baseline triglyceride level of greater than 1000 mg/dl (11.3 mmol/L).

13) A method of treating dyslipidemia in a renally impaired adult patient and a non-renally impaired adult patient comprising administering to both patients 0.2 mg of pemafibrate or a pharmaceutically acceptable salt thereof twice daily.

14) The method of claim 13 wherein the renally impaired patient is mildly to moderately renally impaired.

15) The method of claim 13 wherein the renally impaired patient is severely renally impaired.

16) The method of claim 1 wherein the subject wherein the subject has an HDL-C concentration of less than 40 mg/dL and is on moderate to high intensity statin therapy.

17) The method of claim 1 wherein the subject wherein the subject has an HDL-C concentration of less than 40 mg/dL and has an LDL-C concentration less than 70 mg/dL.

18) The method of claim 1 wherein the subject has an HDL-C concentration of less than 40 mg/dL and is on moderate to high intensity statin therapy and has a triglyceride concentration of from 200 to 500 mg/dL.

19) The method of claim 1 wherein the subject has an HDL-C concentration of less than 40 mg/dL and has an LDL-C concentration less than 70 mg/dL and has a triglyceride concentration of from 200 to 500 mg/dL.

20) The method of claim 1 wherein the method prevents a cardiovascular event selected from nonfatal myocardial infarction, nonfatal ischemic stroke, hospitalization for unstable angina requiring unplanned coronary revascularization, cardiovascular death, and combinations thereof.
